(19) United States
(12) Patent Application Publication
(10) Pub. No.: US 2026/0045329 A1
(43) Pub. Date: Feb. 12, 2026

(54) SYSTEM AND METHOD TO OPTIMIZE TELEPATHOLOGY IMAGE ANALYSIS

(71) Applicant: QTC MANAGEMENT, INC., San Dimas, CA (US)

(72) Inventors: Todd Rothrock Minnigh, Brookfield, WI (US); Larry John Schaefer, San Antonio, TX (US); Shabbir Akbar Bharmal, Ellicott City, MD (US)

(21) Appl. No.: 19/293,344

(22) Filed: Aug. 7, 2025

Related U.S. Application Data
(60) Provisional application No. 63/680,983, filed on Aug. 8, 2024.

(51) Int. Cl. G16H 10/20; G06T 7/00; G06V 10/764; G06V 10/774; G06V 10/82; G16H 70/60

(52) U.S. Cl. CPC G16H 10/20; G06T 7/0012; G06V 10/764; G06V 10/774; G06V 10/82; G16H 70/60; G06T 2207/20081; G06T 2207/20084; G06T 2207/30004; G06V 2201/03

(57) ABSTRACT

Systems and methods for optimizing the performance of telepathology image analysis in a distributed computing environment are discussed.

### RELATED APPLICATION

This application claims the benefit of, and priority to, U.S. Provisional Patent Application No. 63/680,983, filed Aug. 8, 2024, entitled “System and Method to Optimize Telepathology Image Analysis”, the entire content of which is incorporated herein by reference in its entirety.

### BACKGROUND

Pathologists study blood, urine, tissue and other materials removed from a patient to diagnose illness or disease. Today many pathology studies are performed by analyzing digital images of slides of the removed material captured using a whole slide scanner or camera. Digital studies may also be created by scanning existing archives of slides. In most cases slides are labeled with barcode identification for tracking and workflow management. Each of these pathology studies may include multiple images that are 1-2 GB each and each study typically includes five or more images. The images may be analyzed locally at a hospital or via a telepathology network where the images of the material are collected and transmitted to a remote location for analysis.

Large telepathology networks are inherently complex, reaching hundreds of pathology subspecialists, potentially across multiple time zones. Presenting the scanned images to the optimal subspecialist for the most rapid and accurate diagnosis is critical. This can reduce the time to treatment initiation (TTI) for patients with disease, which is highly correlated with better outcomes.

### BRIEF SUMMARY

Embodiments of the present invention provide techniques for programmatically identifying an optimal pathologist or pathologists to read an image study. More particularly, embodiments use an analysis module to analyze images and their associated metadata. This early analysis process, which may employ artificial intelligence, pre-filters images from the study to identify and remove ‘normal’ studies so as to reduce the number of images that need to be read and transmitted. Embodiments also identify an appropriate, and ideally optimal, pathologist to read the images. Embodiments may further consider current pathologist availability and optionally past performance in determining which pathologist should read the image studies.

In one embodiment, a computing device-implemented method optimizes telepathology image analysis using a computing device that includes at least one processor. The method includes receiving a pathology study that includes multiple images and reviewing, with an analysis module executed with the aid of the at least one processor, one or more of metadata or referral notes associated with each of the images to identify one or more of a tissue type and suspected disease or condition. The method further includes performing a programmatic analysis of each of the images in the pathology study with the analysis module based on one or more of the tissue type and suspected disease or condition, Additionally, the method determines, based on the programmatic analysis, that at least one of the plurality of images in the pathology study is considered a suspect image that is not considered pathologically normal. The method additionally programmatically identifies a pathologist to read the pathology study based on the determining that the study includes at least one suspect image and transmits the pathology study containing the at least one suspect image over a network to the identified pathologist for review.

In another embodiment, a computing device-implemented method optimizes telepathology image analysis using a computing device that includes at least one processor. The method includes receiving a pathology study that includes multiple images and reviewing, with an analysis module executed with the aid of the at least one processor, one or more of metadata or referral notes associated with each of the images to identify one or more of a tissue type and suspected disease or condition. The method further includes performing a programmatic analysis of each of the images in the pathology study with the analysis module based on one or more of the tissue type and suspected disease or condition, Additionally, the method determines, based on the programmatic analysis, that a first subset of the images in the pathology study are considered pathologically normal and that a second subset of the plurality of images in the pathology study are considered suspect images that are not considered pathologically normal.

The method also programmatically identifies a pathologist to read the suspect images and transmits over a network only the second subset of the suspect images from the pathology study to the identified pathologist for review.

### BRIEF DESCRIPTION OF THE DRAWINGS

The accompanying drawings, which are incorporated in and constitute a part of this specification, illustrate one or more embodiments of the invention and, together with the description, help to explain the invention. In the drawings:

FIG. 1 depicts an exemplary environment suitable for practicing one or more embodiments of the present invention;

FIG. 2A depicts an exemplary sequence of steps followed by an embodiment of the present invention to optimize telepathology image analysis;

FIG. 2B depicts an exemplary sequence of steps followed by an alternate embodiment of the present invention to optimize telepathology image analysis;

FIG. 3 depicts an exemplary sequence of steps followed by an embodiment of the present invention to programmatically identity an optimal pathologist to read an image study;

FIG. 4 depicts an alternate depiction of a workflow for analyzing pathology image studies in one or more embodiments; and

FIG. 5 depicts an exemplary national telepathology network in an embodiment.

### DETAILED DESCRIPTION

Many digital pathology networks of significant size will have hundreds to thousands of pathologists across a broad spectrum of subspecialties. These pathologists may work in multiple time zones, with widely ranging workload queues. In large distributed networks of this type, knowing what image to direct to which pathologist for a response that is both accurate and rapid is a challenge. The right specialist must be identified, the right images must be selected and the images must be transmitted at a time at which they may acted on in a rapid manner. In smaller networks today this is managed by tribal knowledge and local systems which support manual look-up of credentialed individuals. In a regional, national, or global telepathology network such look-ups are unworkable.

Considering the complexity of a network nationally or globally with multiple time zones, thousands of pathologists, many subspecialties, and perhaps one hundred or more scanning and reading sites, the challenge of where to send each pathology study for interpretation is considerable. As the images are 1-2 GB each with typically five or more images in a study, sending every study to every network node is not practical. Instead an approach is needed to determine where a prioritized study may best be read by an identified pathologist that is both capable of reading the type of study and also available to perform the reading.

Embodiments of the present invention pre-filter the images in pathology studies of a patient before their reading by a selected pathologist with the use of a computing device-executed analysis module. Embodiments use one or more of Artificial Intelligence (AI), logic, credentialing data, and processing latency, to pre-filter images in pathology studies to identify priority images and find the right pathologist(s) that is/are currently available in a network, with the fastest expected response times, to review the images. For example, the analysis module, which may include one or more AI algorithms, may consider metadata associated with the pathology study that indicates the tissue type of the captured sample and any accompanying referral notes which may indicate a suspected disease, illness or condition. The analysis module also performs a preliminary analysis of the images. This analysis is used in part to determine the likelihood of all of the images in the study having a diagnosis of a ‘normal’ sample. Studies designated as ‘normal’ may lead to a lower priority score being assigned for the reading of the study so that it may be de-prioritized for examination.

In some embodiments, the analysis module may further be used to determine or refine a sub-category for the study by disease type and provider and to identify a relevant sub-specialty of pathology for which at least some pathologists on the network are credentialed.

Embodiments may determine the local time of the provider (i.e.: which time zone) and provider availability (i.e. current work load and work hours) from historic performance or integrated data,. For urgent reads in surgery cases, the slide can be subcategorized and flagged “stat/surgery” or similarly marked as urgent as an immediate response may determine the next step in surgery.

The analysis module may identify the historic response times of the qualified pathologists for the type of study, the likelihood they are currently available and available data of their current workload in determining where to send the study to be read. Embodiments may also dispatch the images to one or multiple pathologists for reading. In such a case, following a read by a first pathologist, any others to whom the images were dispatched may be informed of the study completion so that the work is not duplicated. In other embodiments where a second opinion is desired, the system may wait until a second read result is received so the two reads can be compared before informing the other pathologists that the study has been completed.

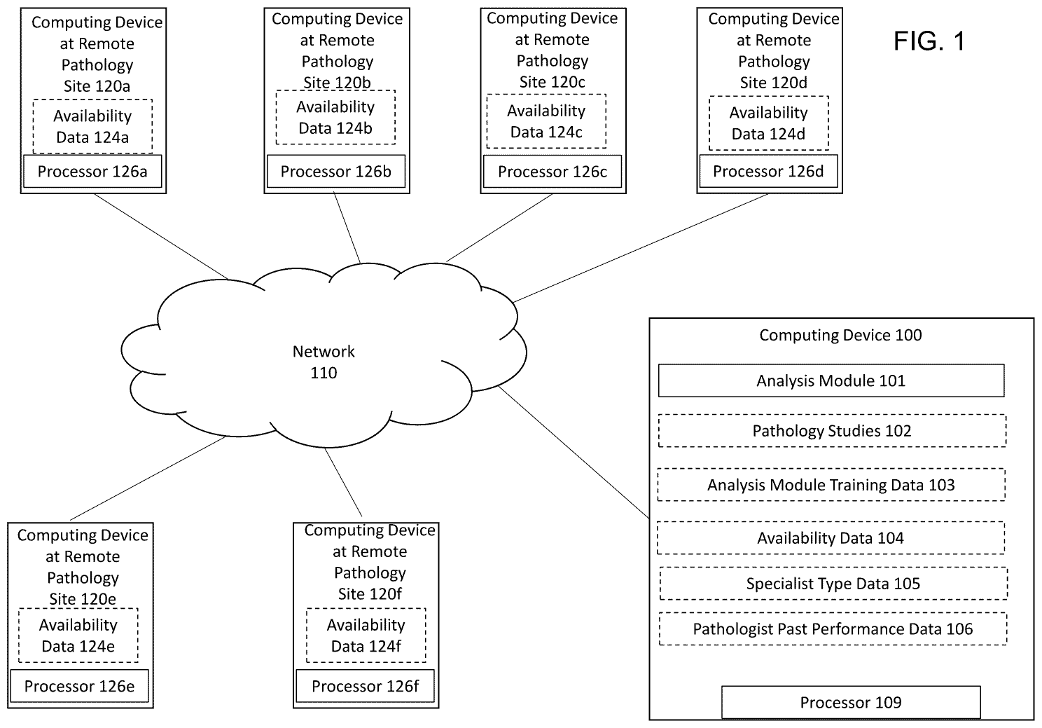

FIG. 1 depicts an exemplary environment suitable for practicing one or more embodiments of the present invention. A network accessible computing device 100 executes an analysis module 101. Analysis module 101 may be implemented in software, hardware or a combination thereof, and may include the use of one or more Graphical Processing Units (GPUs), Field Programmable Gate Arrays (FPGAs) and Application Specific Integrated Circuits (ASICs). Analysis module 101 may include one or more artificial intelligence algorithms performing one or more types of AI. The algorithms may include supervised learning algorithms training a model on pre-classified data such as Neural Networks, Decision Trees, Random Forest linear regression, time-series regression, support vector machines and logistic regression algorithms. Analysis module 101 may include unsupervised learning algorithms where a model is trained on unlabeled data where patterns are identified within the data set during training including algorithms such as clustering algorithms including k-means clustering, data reduction algorithms including Principal Component Analysis (PCA), and auto encoders. Analysis module may also include reinforcement algorithms where the model learns from feedback in an environment such as Asynchronous Actor-Critic Agents (A3C), Q-learning, Deep Adversarial Networks, Monte-Carlo Tree Search (MCTS), SARSA (state-action-reward-state-action) and policy gradients. In an alternative embodiment, analysis module 101 may also utilize Computer Vision in combination with/or instead of AI in programmatically analyzing images in a pathology study.

In one embodiment, analysis module 101 may include an AI model trained using sets of data from previous pathology studies to draw a connection between the images and the presence or absence of disease and particular medical conditions. The data sets may include images of various types of captured blood, urine, tissue and other materials, their related metadata and referral notes, and the conclusions that were ultimately drawn by the pathologist who read the study. Of particular benefit is the ability to rule out normal studies for common pathology types with exceptionally high sensitivity (near zero false negatives). Such an algorithm allows the system to prioritize those studies appropriately and direct resources to studies more likely to prove positive. In one embodiment, the threshold value for the model determining that a study is ‘normal’ (and thus de-prioritizing it from the initial set of studies sent to the pathologist for reading) may be user configurable. In some embodiments the default threshold sensitivity is specifically designed to err on the side of including studies for examination rather than omitting them. However, it will be appreciated that even with such a default threshold that is tilting towards inclusion the ability to de-prioritize provides a demonstrable efficiency benefit both in reducing the amount of data sent over the network and resulting in faster review times by the pathologist who has fewer images to review. In some embodiments the threshold sensitivity for determining whether a study is normal is calibrated to align with clinical objectives, using control samples and expert pathologist inputs for validation. Thresholds are validated statistically and re-validated based on workflow changes with adherence to guidelines from FDA and clinical and laboratory standards initiative (CLSI) It should be further appreciated that any studies initially deprioritized may be stored and examined later or as the need arises.

In an alternate embodiment, a similar but more granular approach may be taken on an image basis within each study. That is, individual images that are initially analyzed and considered normal may be de-prioritized and removed from the study with the remainder of the images, the ‘suspect’ images, being sent as part of the study to the remote pathologist for prioritized examination. The de-prioritized images filtered out of the study may be stored and examined at a later time as needed.

As discussed above and further below, analysis module 101 is used to examine pathology studies including the images of captured patient samples and related metadata and referral notes. Pathology studies 102 may be permanently stored on computing device 101 or may be retrieved from another network accessible location such as a separate database or separate facility. In one embodiment, computing device 100 will be associated with a hospital, clinic or other point of care provider at which the pathology study for a patient is initially captured. Computing device 100 may be a server, desktop, mobile device or some other type of computing device equipped with one or more processors 109 including graphics processing units (GPUs) and/or central processing units (CPUs) and is connected via a network interface (not shown) to network 110 and computing devices at remote pathology sites 120a-f. Network 110 may the Internet, a local area network (LAN), intranet, cellular network or some other type of network that enables communication between computing device 100 and computing devices at remote pathology sites 120a-f.

In one embodiment, computing device 100 may include analysis module training data 103 for training one or more AI models utilized by analysis module 101. In another embodiment, analysis module training data 103 may be located elsewhere and used to remotely train an AI model used by analysis module 101. It should be appreciated that in some embodiments multiple AI models from multiple digital pathology platforms may be used to validate results.

In some embodiments, computing devices at remote pathology sites 120a-f are associated with facilities at which pathologists who are candidates to read the image studies practice. Computing devices at remote pathology sites 120a-f each include one or more processors 126a-f and network interfaces (not shown) for communicating over network 110. In some embodiments, computing devices at remote pathology sites 120a-f may make availability data 124a-f accessible for retrieval by analysis module 101 for determining pathologist availability. Availability data 124a-f may include, without limitation, scheduled work hours and current workload information for the candidate pathologists. As explained in more detail below, analysis module 101 may use availability data 124a-f and a current local time (which becomes important when the network of pathologists span multiple time zones and the analysis module may be located in a different time zone) in determining where to send the pathology studies for reading. In some embodiments, instead of waiting for analysis module 101 to request availability data 124a-f, computing devices at remote pathology sites 120a-f may periodically send the availability data to computing device 100 where it can be accessed locally as availability data 104 by the analysis module.

In some embodiments, computing device 100 includes or provides access to specialist type data 105. Specialist type data 105 includes information about the practices of the different candidate pathologists in the network to whom pathology study 102 may be sent. The information may include experience levels, location and any sub-specialty information for each pathologist. For example, specialist type data 105 may include, without limitation, pathologists available to read images in the fields of Dermatopathology, Hematopathology, Cytopathology, Neuropathology, General Pathology, Breast Specialists, Prostate Specialists, Bone & Soft Tissue Pathology, Breast Pathology, Cardiovascular Pathology, Cytopathology, Dermatopathology, Forensic Pathology, Gastrointestinal/Liver Pathology, General Pathology, Genitourinary Pathology, Gynecologic Pathology, Head & Neck Pathology, Hematopathology, Medical Microbiology, Molecular Pathology, Neuropathology, Pediatric Pathology, Pulmonary Pathology, Renal Pathology or Surgical Pathology or other sub specialties.

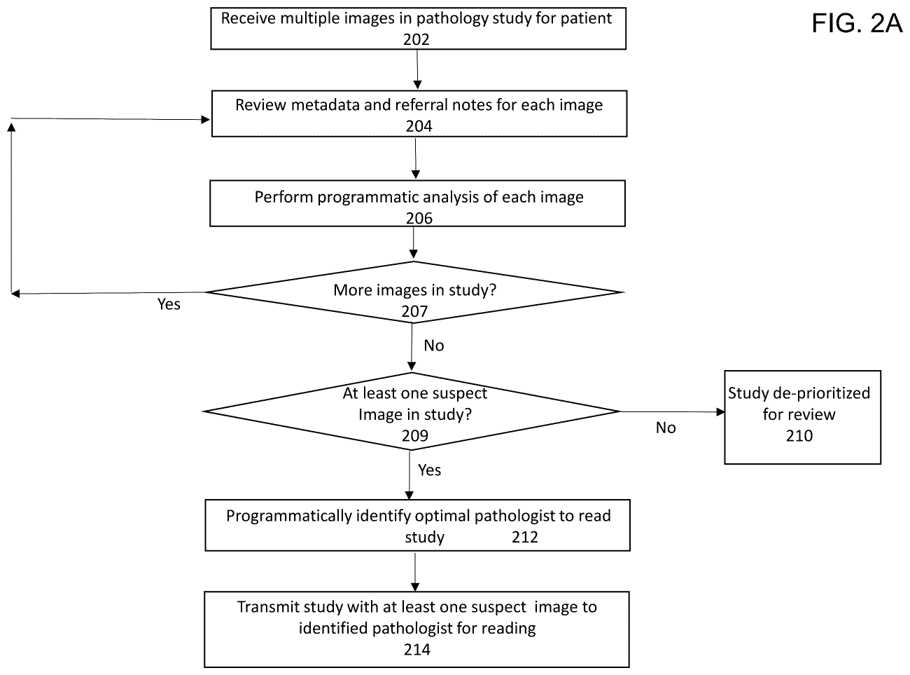

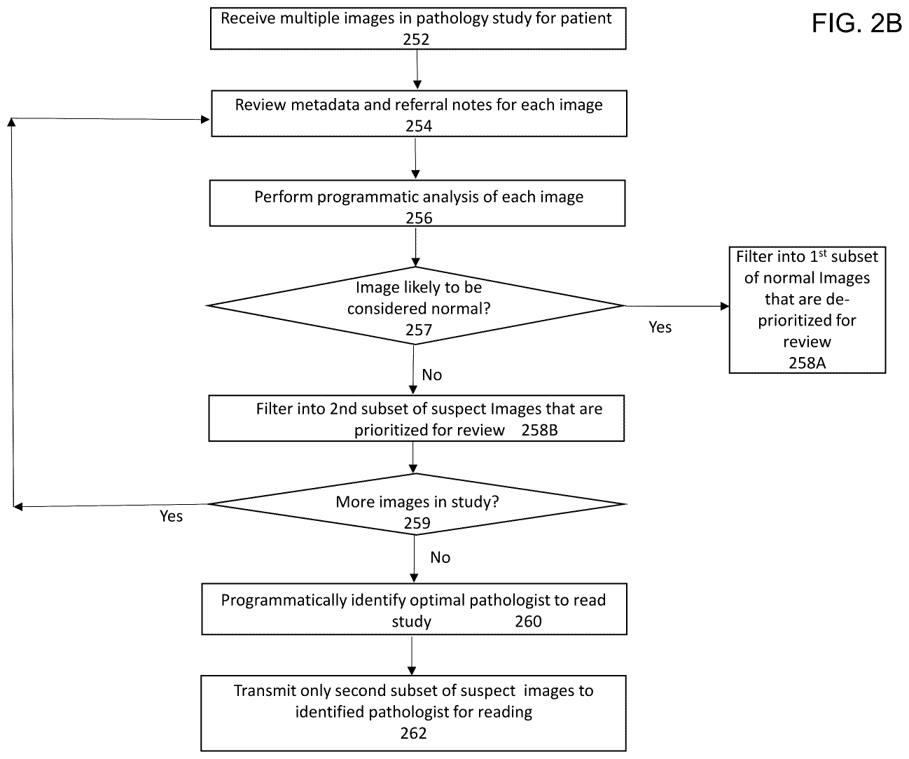

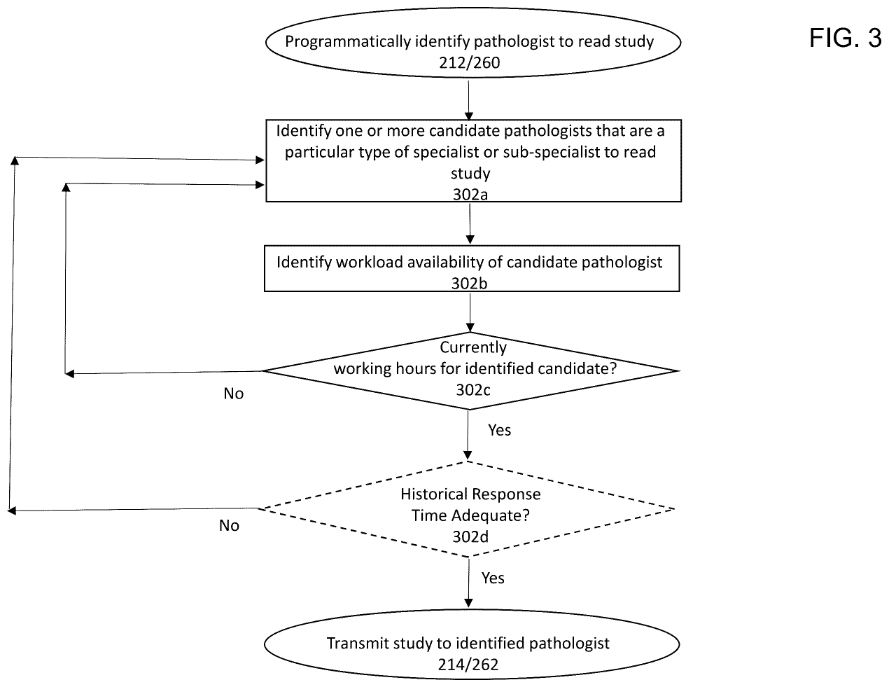

Specialist type data 105 may be analyzed by analysis module 101 as set forth in more detail below with respect to FIG. 2A, FIG. 2B and FIG. 3 to determine where to send pathology study 102 for review.

In some embodiments, computing device 100 includes or provides access to pathologist past performance data 106. Pathologist past performance data 106 includes information about previous response times of the different candidate pathologists in the network to whom pathology studies have been previously sent. The information may be used by analysis module 101 as set forth in more detail below with respect to FIG. 2A, FIG. 2B and FIG. 3 in determining where to send pathology study 102 for review.

FIG. 2A depicts an exemplary sequence of steps followed by an embodiment of the present invention to optimize telepathology image analysis. The sequence begins with the receipt of multiple images in a pathology study 102 for a patient (step 202). The pathology study includes metadata associated with the images and referral notes from the originator of the study. Analysis module 101 reviews the metadata and referral notes for each image (step 204). For example, the metadata may include the tissue type of the imaged sample and the referral notes may indicate a suspected disease or condition and/or contain information about the patient such as, but not limited to, sex, age, ethnicity, and known comorbidities. Analysis module 101 performs a programmatic analysis of each image in the study based on the tissue type and suspected disease or condition to determine whether the image is likely to be found to be a pathologically normal image or whether it is a suspect image showing indicia of the particular disease or condition of interest (step 206). As discussed above, the analysis may be performed with the use of an AI model, computer vision or some other programmatic image recognition process to determine whether or not the image is likely to be considered normal. In some embodiments the programmatic analysis (step 206) may use multiple algorithms searching the image for indicia of different diseases or illnesses. After each examination, a determination is made as to whether there are more images in the study (step 207). If so the process iterates and analysis module 101 reviews the next image. If there are no more images in the study (step 207) a determination is made as to whether at least one suspect image showing indicia of a particular disease or condition of interest was identified in the study (step 209). If not and all of the images in the study were considered to be normal, the study is de-prioritized for review and stored for later examination if necessary (step 210). If there was at least one suspect image identified in the study, the study is prioritized for examination and analysis module 101 programmatically identifies a pathologist to read the remaining suspect images in the study (step 212). An exemplary sequence for programmatically identifying one or more pathologists is discussed in greater detail in FIG. 3. Once the target pathologist(s) has/have been identified, the pathology study is transmitted over the network to one or more selected pathologists to be read (step 214). In this manner, the response time for reading prioritized studies is shortened as studies that contain only normal images are not sent for review and there is accordingly less data to transmit and to review.

By way of further example of the telepathology optimization process, in a first example, a patient may present with a lump in her breast for which treatment protocols indicate that a diagnostic mammogram is indicated. Accordingly, a needle core biopsy may be taken and microscope slides prepared from the tissue samples. The slides are filtered for ‘normal’ samples by an examining algorithm configured with a very high sensitivity that errs on the side of a false positive finding of illness or disease at the expense of a false negative. That is, it is only the samples that are analyzed as clearly negative that are considered normal. The analysis may involve multiple algorithms that separately analyze each image for different diseases or illnesses. In some embodiments, the selection of which algorithm to use for filtering may be made based on an input list of diseases and illnesses that are considered to be possible and need to be ruled out. In other embodiments, all of the available analysis algorithms may be applied in all cases. Using a high threshold sensitivity is an approach that may increase the number of false positives but still reduces the total number of prioritized studies being sent for examination over the network thereby lessening the examination load on the telepathologist doing the reading and the data transmission load of the network over which the studies are sent. Returning to the example, following the reading of the prioritized study, the remote pathologist may determine the patient has a tumor that is a benign fibroadenoma.

In a further example of the telepathology optimization process, a patient may present at a hospital with discomfort from a bulging abdomen. The discomfort may lead to surgery during which a soft tissue mass may be found. A biopsy may be taken during surgery and frozen section slides may be created from the removed material. The surgeon may request a pathology review during the surgery but the on-call pathologist may be unsure of the diagnosis. Following the programmatic analysis of images of the slides that reveal at least one suspect image, an optimal telepathologist who can perform a reading of the images on a ‘stat’ basis may be identified and the images sent to him or her for review. For example, the images may be routed to a pathologist specializing in soft tissue sarcoma and their review may determine the image to reveal a very rare cancer such as an alveolar, soft tissue sarcoma. In this scenario, the surgeon has an answer in less than an hour into surgery thanks to an efficient national/global specialist network and is able to follow an optimal protocol for the disease such as leaving the sarcoma in place, treating it with radiation and then removing the tumor later post tumor regression. This quick pathology result during surgery enables the surgeon to reduce adjacent tissue damage and the likelihood of an inadvertent trigger of metastasis from surgical intervention.

FIG. 2B depicts an exemplary sequence of steps followed by an alternate embodiment of the present invention to optimize telepathology image analysis. The sequence begins with the receipt of multiple images in a pathology study 102 for a patient (step 252). The pathology study includes metadata associated with the images and referral notes from the originator of the study. Analysis module 101 reviews the metadata and referral notes for each image (step 254). For example, the metadata may include the tissue type of the imaged sample and the referral notes may indicate a suspected disease or condition and/or contain information about the patient such as, but not limited to, sex, age, ethnicity, and known comorbidities.

Analysis module 101 performs a programmatic analysis of each image in the study based on the tissue type and suspected disease or condition to determine whether the image is likely to be found to be a pathologically normal image or whether it is a suspect image showing indicia of the particular disease or condition of interest (step 256). As discussed above, the analysis may be performed with the use of an AI model, computer vision or some other programmatic image recognition process to determine whether or not the image is likely to be considered normal. In some embodiments the programmatic analysis (step 256) may use multiple algorithms searching the image for indicia of different diseases or illnesses. If analysis module 101 determines the image is likely to be considered normal (step 207), the analysis filters the image into a first pathologically ‘normal’ subset of images (step 258A). If analysis module 101 determines the image is not likely to be considered normal (step 207) and instead is therefore a ‘suspect’ showing indicia of a disease, illness or condition of interest, the image is filtered into a second subset of ‘suspect’ images that are suspected to not be pathologically normal (step 258B). Analysis module 101 determines if there are more images in the pathology study (step 259) and if so the sequence iterates. If the image just reviewed is the last image in the study (step 259), analysis module 101 programmatically identifies a pathologist to read the remaining suspect images in the study (step 260). An exemplary sequence for programmatically identifying one or more pathologists is discussed in greater detail in FIG. 3. Once the target pathologist(s) has/have been identified, only the second subset of the suspect (pathologically non-normal) images that remain in the pathology study after filtering out the “normal” images are transmitted over the network to one or more selected pathologists to be read (step 262). In this manner, the response time for reading the studies is shortened as the identified likely normal images are not included in the study and there is less data to transmit and to review.

FIG. 3 depicts an exemplary sequence of steps followed by an embodiment of the present invention to programmatically identity one or more hopefully optimal pathologists to read a pathology study as indicated in steps 212 and 260 of FIGS. 2A and 2B respectively. The sub-sequence begins with analysis module 101 identifying one or more candidate pathologists that are specialists or sub-specialist of a particular type needed to review the study (step 302a) using the information from the metadata, referral notes and image analysis in combination with specialist type data 105. Analysis module 101 then identifies workload availability of the identified candidates (step 302b). For example, based on pre-defined criteria, pathologists who have more than x studies scheduled to be read in a pre-defined time period may be filtered out as candidates. Analysis module 101 may also check to see if it is currently working hours for the candidate pathologists (step 302c) which is a concern for emergency reads and may impact who is chosen to read the study for both those urgent reads and/or normal reads. For example, if the pathology study originates in a hospital in Hawaii but the candidate pathologist is on the East Coast of the United States and has just signed off for the day, that candidate may be offline for 12-16 hours depending on work schedule. In such a case, pre-defined criteria may direct analysis module 101 to omit that candidate pathologist from consideration and direct the study to another candidate whose work day is just beginning and would be more likely to start on reading the study sooner. In some embodiments, analysis module 101 may also consider Pathologist Past Performance Data 106 to ensure that only candidate pathologists whose previous performance meets specified benchmarks (e.g. performed a read within a specified time frame such as two, four, six or eight hours) for certain types of studies are selected (step 302d). In such a case, where multiple candidate pathologists have been identified as able to read the study, analysis module 101 may iterate the sequence and consider and eventually send the study to a different candidate pathologist. Once a candidate pathologist has been identified and meets the workload, working hours and past response time requirements, the study may be transmitted to the candidate pathologist as outlined in steps 214 and 262 of FIGS. 2A and 2B respectively. As noted previously, in some circumstances more than one pathologist may meet the criteria and the study may be sent to multiple pathologists in parallel or in sequence depending on circumstances.

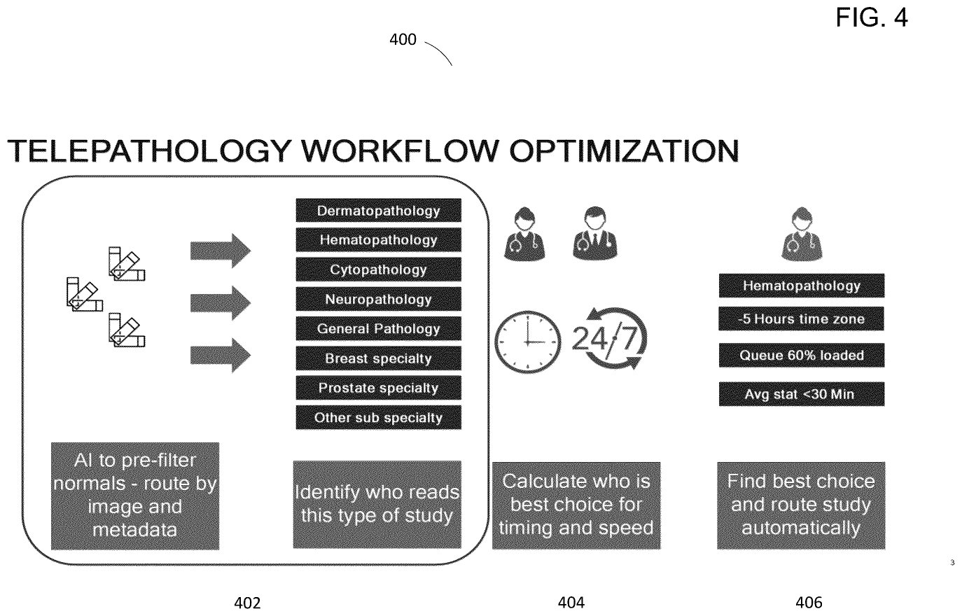

FIG. 4 depicts an alternate depiction of a workflow 400 for analyzing pathology image studies in one or more embodiments. An AI model is used to pre-filter normal studies out and the metadata for the images is examined to identify the relevant specialty/sub-specialty for the pathology study (step 402). Pathologist availability and performance data is reviewed as discussed above to determine a best choice amongst the group of identified specialists (step 404). Once identified the study is routed to the one or more pathologists that meet all of the criteria (step 406). For example, in an optimal case, the pathologist will have the right experience, a light workload, be currently working and have historically excellent response times.

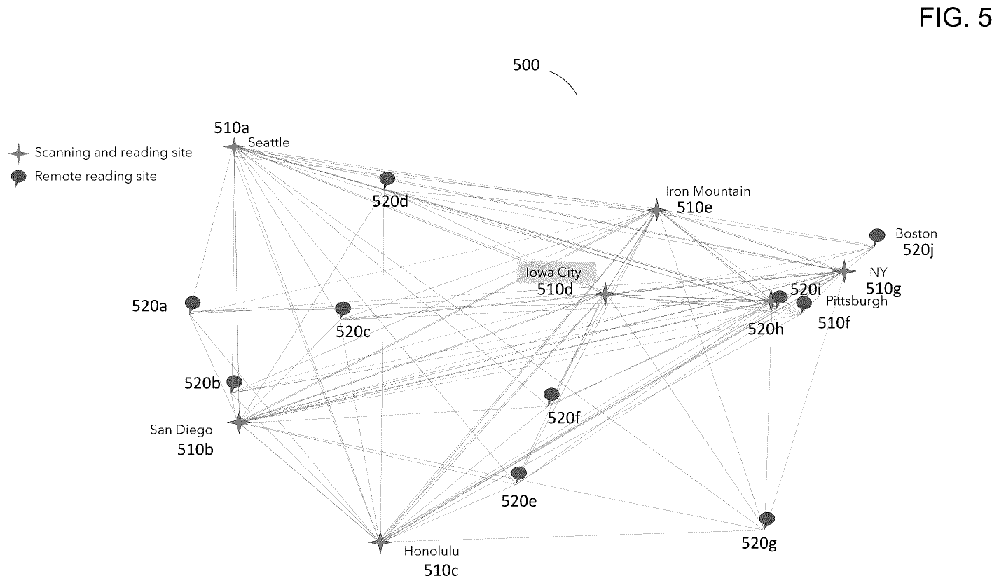

FIG. 5 depicts an exemplary telepathology network 500 in an embodiment. The exemplary telepathology network 500 includes seven scanning and reading sites 510a-510f, and ten remote reading (only) sites 520a-520j. Of note each of the scanning and reading sites connects to all of the other sites and the network spans six separate time zones. In some embodiments, certain nodes may be designated as conduits leading to reading sites for additional specialties outside telepathology network 500. As described herein, telepathology network 500 may provide pathology study reading coverage by time zone, may increase specialist access and may balance workload among local and remote sites. It should be appreciated that FIG. 5 is exemplary rather than limiting as telepathology network 500 may include a greater or lesser number of scanning and/or reading sites than depicted.

Portions or all of the embodiments of the present invention may be provided as one or more computer-readable programs or code embodied on or in one or more non-transitory mediums. The mediums may be, but are not limited to a hard disk, a compact disc, a digital versatile disc, a flash memory, a PROM, a RAM, a ROM, or a magnetic tape. In general, the computer-readable programs or code may be implemented in many computing languages.

Since certain changes may be made without departing from the scope of the present invention, it is intended that all matter contained in the above description or shown in the accompanying drawings be interpreted as illustrative and not in a literal sense. Practitioners of the art will realize that the sequence of steps and architectures depicted in the figures may be altered without departing from the scope of the present invention and that the illustrations contained herein are singular examples of a multitude of possible depictions of the present invention.

The foregoing description of example embodiments of the invention provides illustration and description, but is not intended to be exhaustive or to limit the invention to the precise form disclosed. Modifications and variations are possible in light of the above teachings or may be acquired from practice of the invention. For example, while a series of acts has been described, the order of the acts may be modified in other implementations consistent with the principles of the invention. Further, non-dependent acts may be performed in parallel.

We claim:

1. A computing device-implemented method to optimize telepathology image analysis, the computing device including at least one processor, the method comprising:
receiving a pathology study that includes a plurality of images;
reviewing, with an analysis module executed with the aid of the at least one processor, one or more of metadata or referral notes associated with each of the plurality of images to identify one or more of a tissue type and suspected disease or condition;
performing a programmatic analysis of each of the plurality of images in the pathology study with the analysis module based on the identified one or more of the tissue type and suspected disease or condition;
determining based on the programmatic analysis, that at least one of the plurality of images in the pathology study is considered a suspect image that is not considered pathologically normal;
programmatically identifying, based on the determining that the study includes at least one suspect image, a pathologist to read the pathology study; and
transmitting the pathology study containing the at least one suspect image over a network to the identified pathologist for review.

2. The method of claim 1 wherein the analysis module includes an artificial intelligence (AI) model.

3. The method of claim 2, wherein the AI model is a machine learning model and further comprising:
training the AI model using a data set of previously classified images of a plurality of tissue types associated with a plurality of conditions and/or diseases and their associated metadata, and a data set of previously classified referral notes associated with the previously classified images; and
performing the programmatic analysis using the trained AI model.

4. The method of claim 2 wherein the analysis module executes multiple different algorithms to perform the programmatic analysis of each of the plurality of images.

5. The method of claim 2 wherein the AI model is a neural network.

6. The method of claim 1, wherein the pathologist is identified based at least in part on pathologist specialty.

7. The method of claim 1, wherein the pathologist is identified based at least in part on pathologist availability.

8. The method of claim 7 wherein the pathologist availability is based on one or more of a current time in a local time zone of the pathologist, a current work queue of the pathologist and/or a historical response time of the pathologist.

9. The method of claim 1, wherein the pathologist is identified at least in part based on availability of network conditions for image transmission of the suspect images to the pathologist.

10. The method of claim 1, further comprising:
transmitting the second subset of studies with suspect images over the network to the identified pathologist during a patient's surgery.

11. A non-transitory medium holding processor executable instructions for optimizing telepathology image analysis, the instructions when executed causing at least one computing device equipped with one or more processors to:
receive a pathology study that includes a plurality of images;
review one or more of metadata or referral notes associated with each of the plurality of images to identify one or more of a tissue type and suspected disease or condition;
perform a programmatic analysis of each of the plurality of images in the pathology study based on the identified one or more of the tissue type and suspected disease or condition;
determine based on the programmatic analysis, that at least one of the plurality of images in the pathology study is considered a suspect image that is not considered pathologically normal;
programmatically identify, based on the determining that the study includes at least one suspect image, a pathologist to read the pathology study; and
transmit the pathology study containing the at least one suspect image over a network to the identified pathologist for review.

12. The medium of claim 11 wherein the programmatic analysis is performed using an artificial intelligence (AI) model.

13. The medium of claim 12, wherein the AI model is a machine learning model and the instructions when executed further:
train the AI model using a data set of previously classified images of a plurality of tissue types associated with a plurality of conditions and/or diseases and their associated metadata, and a data set of previously classified referral notes associated with the previously classified images; and
perform the programmatic analysis using the trained AI model.

14. The medium of claim 12 wherein multiple different algorithms are executed to perform the programmatic analysis of each of the plurality of images.

15. The medium of claim 12 wherein the AI model is a neural network.

16. The medium of claim 11, wherein the pathologist is identified based at least in part on pathologist specialty.

17. The medium of claim 11 wherein the pathologist is identified as available at least in part based on one or more of a current time in a local time zone of the pathologist, a current work queue of the pathologist and/or a historical response time of the pathologist.

18. The medium of claim 11, wherein the pathologist is identified at least in part based on availability of network conditions for image transmission of the suspect images to the pathologist.

19. The medium of claim 11, wherein the instructions when executed further cause the at least one computing device to:
transmit the second subset of suspect images over the network to the identified pathologist during a patient's surgery.

20. A computing device-implemented method to optimize pathology image analysis, the computing device including at least one processor, the method comprising:
receiving a pathology study that includes a plurality of images;
reviewing, with an analysis module executed with the aid of the at least one processor, one or more of metadata or referral notes associated with each of the plurality of images to identify one or more of a tissue type and suspected disease or condition;
performing a programmatic analysis of each of the plurality of images in the pathology study with the analysis module based on the identified one or more of the tissue type and suspected disease or condition;
determining based on the programmatic analysis, that a first subset of the plurality of images in the pathology study are considered pathologically normal;
determining, based on the programmatic analysis, that a second subset of the plurality of images in the pathology study are considered suspect images that are not considered pathologically normal;
programmatically identifying a pathologist to read the suspect images; and
transmitting over a network only the second subset of the suspect images from the pathology study to the identified pathologist for review.
